(19) United States
(12) Patent Application Publication
(10) Pub. No.: US 2023/0330037 A1
(43) Pub. Date: Oct. 19, 2023

(54) METHOD AND COMPOSITION FOR PREVENTING AND TREATING VIRAL RESPIRATORY INFECTIONS

(71) Applicant: Bright Green Corporation, Wilmington, DE (US)

(72) Inventor: V. Hari, Orlando, FL (US)

(21) Appl. No.: 17/720,769

(22) Filed: Apr. 14, 2022

(51) Int. Cl. A61K 36/185; A61K 31/05; A61K 36/9066; A61K 36/67

(52) U.S. Cl. CPC A61K 31/05; A61K 36/185; A61K 36/67; A61K 36/9066

(57) ABSTRACT

A method of treating or preventing respiratory viral infections. The method includes administering to a patient a pharmaceutical composition comprising one or more non psychoactive cannabinoid-containing compositions consisting essentially of a cannabinoid fraction including at least one of Cannabigerol (CBG) or Cannabigerolic acid (CBGA) and a non-cannabinoid fraction from Cannabis sativa L., turmeric, and black pepper and other plant sources.

### BACKGROUND

The present invention relates to the use of non-psychoactive formulations for the prevention and/or treatment of infections, such as viral and/or bacterial and other microbial respiratory infections. These formulations may also find use in the treatment of genetic, environmental and other causes that affect not only the respiratory tract but also other body organs and health of humans and animals.

### SUMMARY

In one exemplary embodiment, a method of treating or preventing respiratory viral infections. The method includes administering to a patient a pharmaceutical composition comprising one or more cannabinoid-containing compositions consisting essentially of a cannabinoid fraction including at least one of Cannabigerol (CBG) or Cannabigerolic acid (CBGA) and a non-cannabinoid fraction from Cannabis sativa L., turmeric, and black pepper.

In another embodiment according to any of the previous embodiments, the respiratory viral infections include at least one of any and all strains of SARS-CoV-2 (Covid-19), Respiratory Syncytial virus (“RSV”), Middle Eastern Respiratory Syndrome virus (“MERS-CoV”), or any strain of Orthomyxoviridae.

In another embodiment according to any of the previous embodiments, the cannabinoid containing fraction includes other unnamed non-psychoactive cannabinoids from any cannabinoid containing plants chiefly Cannabis sativa including C. Indica, C. Ruderalis and their hybrids and various sub species and strains and other plants containing cannabinoids.

In another embodiment according to any of the previous embodiments, the non-cannabinoid containing fraction includes at least one or more of terpenes, sterols, triglycerides, alkanes, squalling, tocopherols, carotenoids, chlorophyll, flavonoid glycosides, alkaloids, or other phytochemicals.

In another embodiment according to any of the previous embodiments, the cannabinoid-containing fraction includes other un-named non-psychoactive cannabinoids from any source.

In another embodiment according to any of the previous embodiments, the non-cannabinoid containing fraction includes at least one or more of terpenes, sterols, triglycerides, alkanes, squalling, tocopherols, carotenoids, chlorophyll, flavonoid glycosides, alkaloids, or other phytochemicals from any source.

In another embodiment according to any of the previous embodiments, the cannabinoid-containing fraction is Cannabigerol (CBG), Cannabigerolic acid (CBGA), and unnamed non-psychoactive cannabinoids from any plant or synthetic source.

In another embodiment according to any of the previous embodiments, the non-cannabinoid containing fraction includes at least one or more of terpenes, sterols, triglycerides, alkanes, squalling, tocopherols, carotenoids, chlorophyll, flavonoid glycosides, alkaloids, or other phytochemicals from any source.

In another embodiment according to any of the previous embodiments, the cannabinoid-containing fraction includes a combination of a Cannabigerol (CBG) containing extract and Cannabigerolic acid (CBGA) containing extract.

In another embodiment according to any of the previous embodiments, the cannabinoid-containing fraction is present as one or more cannabis-based medicine extracts (CBME).

In another embodiment according to any of the previous embodiments, the CBME include at least one of Cannabigerol (CBG), Cannabigerolic acid (CBGA), or other un-named cannabinoids.

In another embodiment according to any of the previous embodiments, the non-cannabinoid containing fraction is present as one or more cannabis-based medicine extracts (CBME) including terpenes, alkaloids, sterols, triglycerides, alkanes, squalling, tocopherols, carotenoids, chlorophyll, flavonoid glycosides, or other phytochemicals from any source.

In another embodiment according to any of the previous embodiments, the formulation contains at least one cannabinoid-containing fraction includes Cannabigerol (CBG), Cannabigerolic acid (CBGA), and one or more other minor plant derived components.

In another embodiment according to any of the previous embodiments, the non-cannabinoid containing fraction includes at least one or more of terpenes, sterols, triglycerides, alkanes, squalling, tocopherols, carotenoids, chlorophyll, flavonoid glycosides, or alkaloids from any source.

In another exemplary embodiment, an oro-nasal composition consisting essentially of a cannabinoid fraction including at least one of Cannabigerol (CBG) or Cannabigerolic acid (CBGA) and a non-cannabinoid fraction from Cannabis sativa L., turmeric, and black pepper.

In another embodiment according to any of the previous embodiments, the cannabinoid containing fraction includes other unnamed non-psychoactive cannabinoids from any cannabinoid containing plants chiefly Cannabis sativa including C. Indica, C. Ruderalis and their hybrids and various sub species and strains and other plants containing cannabinoids.

In another embodiment according to any of the previous embodiments, the oro-nasal composition includes at least one of a beverage formulation, mouth sprays or drops, lozenges, gummies, candies, nasal sprays or drops, or liquid capsules.

In another embodiment according to any of the previous embodiments, the non-cannabinoid containing fraction includes at least one or more of terpenes, sterols, triglycerides, alkanes, squalling, tocopherols, carotenoids, chlorophyll, flavonoid glycosides, alkaloids, or other phytochemicals.

In another embodiment according to any of the previous embodiments, the cannabinoid containing fraction and the non-cannabinoid fraction are plant based.

In another embodiment according to any of the previous embodiments, at least one of the cannabinoid containing fraction and the non-cannabinoid fraction may be are synthetic.

### DETAILED DESCRIPTION

This disclosure is directed to a pharmaceutical composition for the prevention and/or treatment of viral infections, such as those caused by SARS-CoV-2 (“Covid-19”), Respiratory Syncytial virus (RSV), Middle Eastern Respiratory Syndrome virus-MERS-CoV, Orthomyxoviridae containing various strains (including H1N1 influenza, H5N1 influenza, Swine and Bird flu) causing influenza and influenza-like symptoms in humans, other mammals, birds and even invertebrates. All these are enveloped viruses with spike-like proteins which attach to receptors in the oro-nasal and naso-pharyngeal regions and enter susceptible individuals causing severe respiratory distress and other symptoms which can be fatal. The disclosed pharmaceutical composition can also be used for the treatment and/or prevention of bacterial infections as well as genetic, environmental and idiopathic causes that could cause respiratory distress.

This disclosure prevents and/or treats the above identified infections and medical conditions by administering to a patient the pharmaceutical composition. The pharmaceutical composition can be administered by oro-nasal drops, oro-nasal sprays, oral liquid capsules, or beverages containing the composition. In one example, the pharmaceutical composition includes at least one or more of a cannabinoid containing fraction and a non-cannabinoid containing fraction. The cannabinoid containing fraction includes at least one of Cannabigerol (CBG) and Cannabigerolic acid (CBGA). The cannabinoid containing fraction may also include one or more non-psychoactive cannabinoid-containing plant fraction and a non-cannabinoid containing fraction.

The Cannabigerol and Cannabigerolic acid are derived from young Cannabis sativa, C. Indica, C. Ruderalis and their hybrids and various sub species and strains. Alternatively, the source of Cannabegerol, Cannabegerolic acid and any other cannabinoid component of the formulation may be from any other source including synthetic derivatives when such synthetic sources are generated. The non-cannabinoid containing fraction can include terpenes and other phytochemicals from C. Indica, C. Ruderalis and their hybrids and various sub species and strains as well as other plant and synthetic sources. The pharmaceutical composition can also include one or more cannabinoid-containing plant extracts.

Cannabis sativa including C. Indica, C. ruderalis and their various sub species, varieties, hybrids, and eco-bio types contain different quantities of about 113 Phyto-Cannabinoids and several terpenes and other phytochemicals. Among these are the psychoactive tetrahydrocannabinol (THC) and non-psychoactive cannabidiol (CBD), as well as other minor Cannabinoids. The disclosed compositions for inhibition of diseases caused by the above-named respiratory viruses will not contain either THC or CBD. THC and CBD possess anti-inflammatory responses that could lead to immunosuppression.

The non-cannabinoid containing fraction can come from a Cannabis sp. plant and its various sub species and strains or other plant and synthetic sources. The non-cannabinoid containing fraction includes at least one of terpenes, sterols, triglycerides, alkanes, squalling, tocopherols, carotenoids, chlorophyll, flavonoid glycosides, alkaloids, or other phytochemicals In addition to the above identified non-cannabinoid containing substances, the pharmaceutical composition may include phytochemicals from Turmeric (Curcuma longa) and/or Black pepper (Piper nigrum).

Cannabigerolic acid (CBGA) is a main one of the cannabinoids because decarboxylation of CBGA results in the generation of the “big six” most widely-researched cannabinoids - tetrahydrocannabinol (THC), cannabidiol (CBD), Cannabichromene (CBC), cannabinol (CBN), tetrahydrocannabivarin (THCV), or Cannabigerol (CBG). All of these cannabinoids start out as CBGA before they’re transformed by enzymes within the Cannabis sp. plant’s trichomes. As this reaction occurs, the CBGA synthesizes into THCA, CBDA, CBCA, and so on. During a typical flowering cycle, this process usually takes six to eight weeks. Cannabigerol is found in higher concentrations in young plants.

Cannabigerol (CBG acts as a cannabinoid receptor type 1 (CB1) antagonist, helping prevent some of the high (and associated anxiety) created by THC. It may also boost levels of anandamide, a natural “bliss molecule” found in the brain that regulates things such as appetite, sleep, and mood. It’s likely that CBG helps block serotonin receptors, making it a potentially useful antidepressant. CBG also works as a Gamma-aminobutyric acid (GABA) uptake inhibitor, meaning that it may help reduce anxiety and relax muscle tension. Some studies report that CBG and another minor cannabinoid cannabinol or have other properties including inhibition of Methicillin resistant bacteria or other un-named cannabinoids.

In one example, the disclosed composition does not contain any psychoactive controlled substances, such as THC or non-psychoactive Cannabidiol (CBD), but will contain Cannabigerol and Cannabigerolic acid and other potential cannabinoids. The composition may contain terpenes and other phytochemicals from Cannabis and other sources as well as Curcumin from Turmeric and piperine from piper nigrum (Black Pepper). One feature of terpenes are biocidal properties. Curcumin from Curcuma longa (turmeric) can also provide anti-viral and multiple other properties and piperine enhances the entry of many phytochemicals into cells.

Thus, CBGA, CBG, some minor cannabinoids like Cannabinol, terpenes, phytochemicals from Turmeric and Black pepper have the feature of providing multiple health related properties especially biocidal and anti-viral properties.

The composition of this disclosure can be administered as beverage formulations, mouth and/or nasal sprays or drops, oro-nasal hygiene products, lozenges, Gums, liquid capsules, oral suspensions, oral preparations, or candies or systemically as liquid capsules or other similar applications.

The composition aids in prevention and treatment of infection by SARS-CoV-2 (Covid-19) and other respiratory viruses such as Respiratory Syncytial virus (RSV), Middle Eastern Respiratory Syndrome virus-MERS-CoV, the Orthomyxoviridae consisting of the flu viruses including H1N1 influenza, H5N1 influenza, Swine and Bird flu and other respiratory viral and bacterial infections. The composition can include Cannabigerol (CBG), Cannabigerolic acid (CBGA) and other non-psychoactive minor cannabinoids with or without one or more non cannabinoids chiefly terpenes and terpenoids from Cannabis including resin, oil from Cannabis species, other medicinal plants and other phytochemicals from Turmeric (Curcuma longa) and Black pepper (Piper nigrum). These formulations may also have the potential as therapeutic agents for medical conditions caused by non-microbial sources.

In one example, a ratio of the cannabinoid-containing fraction to the non-cannabinoid containing fraction is varied. In another example, the cannabinoid containing fraction is greater than the non-cannabinoid containing fraction and in yet another example the cannabinoid containing fraction is less than the non-cannabinoid containing fraction. In one example, the cannabinoid-containing fraction is Cannabigerol (CBG) and Cannabigerolic acid (CBGA) without any other additives and in another example, the cannabinoid-containing fraction is a mixture of Cannabigerol (CBG) and Cannabigerolic acid (CBGA) with a non-cannabinoid containing fraction, such as one or more terpenes and other phytochemicals from both Cannabis, other plants, synthetic sources, turmeric, and black pepper.

The cannabinoid-containing fraction can include one or more non-psychoactive cannabis-based medicine extracts (CBME) besides Cannabigerol (CBG) and Cannabigerolic acid (CBGA) with one or more non-cannabinoid phytochemical extracts from Cannabis as well as Turmeric and Black pepper and other plants.

In this application the term Black pepper includes any and all species of the genus Piper and the term Turmeric includes any and all species, sub species and varieties of the genus Curcuma.

Although the different non-limiting examples are illustrated as having specific components, the examples of this disclosure are not limited to those particular combinations. It is possible to use some of the components or features from any of the non-limiting examples in combination with features or components from any of the other non-limiting examples.

It should be understood that like reference numerals identify corresponding or similar elements throughout the several drawings. It should also be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claim should be studied to determine the true scope and content of this disclosure.

What is claimed is:

1. A method of treating or preventing respiratory viral infections, the method comprising:
administering to a patient a pharmaceutical composition comprising one or more cannabinoid-containing compositions consisting essentially of:
a cannabinoid fraction including at least one of Cannabigerol (CBG) or Cannabigerolic acid (CBGA); and
a non-cannabinoid fraction from Cannabis sativa L., other plants, turmeric, and black pepper.

2. The method of claim 1, wherein the respiratory viral infections include at least one of SARS-CoV-2 (Covid-19), Respiratory Syncytial virus (“RSV”), Middle Eastern Respiratory Syndrome virus (“MERS-CoV”), or Orthomyxoviridae containing strains.

3. The method of claim 1, wherein the cannabinoid containing fraction includes other unnamed non-psychoactive cannabinoids from any cannabinoid containing plants chiefly Cannabis sativa including C. Indica, C. Ruderalis and their hybrids and various sub species and strains and other plants containing cannabinoids.

4. The method of claim 1, wherein the non-cannabinoid containing fraction includes at least one of terpenes, sterols, triglycerides, alkanes, squalling, tocopherols, carotenoids, chlorophyll, flavonoid glycosides, alkaloids, or other phytochemicals.

5. The method of claim 1, wherein the cannabinoid-containing fraction includes other un-named non-psychoactive cannabinoids from any source.

6. The method of claim 5, wherein the non-cannabinoid containing fraction includes at least one of terpenes, sterols, triglycerides, alkanes, squalling, tocopherols, carotenoids, chlorophyll, flavonoid glycosides, alkaloids, or other phytochemicals from any source.

7. The method of claim 1, wherein the cannabinoid-containing fraction is Cannabigerol (CBG), Cannabigerolic acid (CBGA), and unnamed non-psychoactive cannabinoids from any plant or synthetic source.

8. The method of claim 7, wherein the non-cannabinoid containing fraction includes at least one of terpenes, sterols, triglycerides, alkanes, squalling, tocopherols, carotenoids, chlorophyll, flavonoid glycosides, alkaloids, or other phytochemicals from other plants, synthetic sources, or any other source.

9. The method of claim 1, wherein the cannabinoid-containing fraction includes a combination of a Cannabigerol (CBG) containing extract and Cannabigerolic acid (CBGA) containing extract.

10. The method of claim 1, wherein the cannabinoid-containing fraction is present as one or more cannabis-based medicine extracts (CBME).

11. The method of claim 10, wherein the CBME include at least one of Cannabigerol (CBG), Cannabigerolic acid (CBGA), or other un-named cannabinoids.

12. The method of claim 11, wherein the non-cannabinoid containing fraction is present as one or more cannabis-based medicine extracts (CBME) including terpenes, alkaloids, sterols, triglycerides, alkanes, squalling, tocopherols, carotenoids, chlorophyll, flavonoid glycosides, or other phytochemicals from any source.

13. The method of claim 1, wherein the at least one cannabinoid-containing fraction includes Cannabigerol (CBG), Cannabigerolic acid (CBGA), and one or more other minor plant derived components.

14. The method of claim 13, wherein the non-cannabinoid containing fraction includes at least one of terpenes, sterols, triglycerides, alkanes, squalling, tocopherols, carotenoids, chlorophyll, flavonoid glycosides, or alkaloids from any source.

15. An oro-nasal composition, consisting essentially of:
a cannabinoid fraction including at least one of Cannabigerol (CBG) or Cannabigerolic acid (CBGA); and
a non-cannabinoid fraction including Cannabis sativa L., turmeric, and black pepper.

16. The composition of claim 15, wherein the cannabinoid containing fraction includes other unnamed non-psychoactive cannabinoids from any cannabinoid containing plants chiefly Cannabis sativa including C. Indica, C. Ruderalis and their hybrids and various sub species and strains and other plants containing cannabinoids.

17. The composition of claim 15, wherein the oro-nasal composition includes at least one of a beverage formulation, mouth sprays or drops, lozenges, gummies, candies, nasal sprays or drops, or liquid capsules.

18. The composition of claim 15, wherein the non-cannabinoid containing fraction includes at least one of terpenes, sterols, triglycerides, alkanes, squalling, tocopherols, carotenoids, chlorophyll, flavonoid glycosides, alkaloids, or other phytochemicals.

19. The composition of claim 15, wherein the cannabinoid containing fraction and the non-cannabinoid fraction are plant based.

20. The composition of claim 15, wherein at least one of the cannabinoid containing fraction and the non-cannabinoid fraction may be synthetic.
